(19) United States
(12) Patent Application Publication
(10) Pub. No.: US 2001/0056101 A1
(43) Pub. Date: Dec. 27, 2001

(54) USE OF DOPAMINE RECEPTOR ANTAGONISTS IN COMBINATION WITH PARTIAL DOPAMINE AGONIST TO PREVENT TOLERANCE IN TREATING NERVOUS DISORDERS RELATED TO DOPAMINE DYSFUNCTION

(76) Inventors: CAROL A. TAMMINGA, POTOMAC, MD (US); ARVID CARLSSON, GOTHENBERG (SE)

Correspondence Address: VENABLE, PO BOX 34385, WASHINGTON, DC, 200439998

(21) Appl. No.: 09/340,685

(22) Filed: Jun. 29, 1999

Related U.S. Application Data
(60) Provisional application No. 60/090,975, filed on Jun. 29, 1998.

(51) Int. Cl. A61K 31/47; A61K 31/445

(52) U.S. Cl. 514/310

(57) ABSTRACT

Dopamine receptor antagonists are commonly prescribed for the treatment of schizophrenia and psychosis. While they are effective antipsychotics, they fail to treat other aspects of the disorder (e.g. negative symptoms, attention, and concentration) and have severe side effects, ranging from parkinsonism, acute motor side effects, akasthisia, dysphoria, and tardive dyskinesia. Dopamine agonist drug treatments are effective in treating both positive and negative symptoms without the common side effects. Unfortunately, dopamine agonists suffer from efficacy tolerance, the time limited effect on the order of 1-7 days. Thus, despite the partial effectiveness of current treatments, pressing need exists for new treatments. The combination drug therapy described herein meets this need. Specifically, the invention involves the use of small doses of dopamine receptor antagonists to reduce the intrinsic activity of a partial agonist, thereby reversing the agonist-induced tolerance commonly reported with agonist therapy alone.

### FIELD OF THE INVENTION

The field of this invention generally relates to drug therapy for central nervous system disorders involving the administration of receptor agonists of a transmitter, especially partial receptor agonists, in combination with receptor antagonists of the same transmitter to overcome efficacy, tolerance, and side effect problems associated with either agent alone. The field of this invention specifically relates to a treatment for disorders stemming from dopamine dysfunction, such as psychosis, schizophrenia, and movement disorders. This treatment involves the administration of a full or partial dopamine agonist in combination with a dopamine antagonist to prevent tolerance or side effects associated with dopamine antagonist or agonist therapy. Such a treatment scheme can modify the agonist effect and address the active and residual symptoms and side effects associated with dopamine antagonist therapy alone.

### TECHNOLOGY REVIEW

Many diseases and dysfunctional syndromes of the central nervous system are caused by imbalances in neurotransmitters and receptors within the nervous system. Such diseases are often recognized by the accompanying mental or physical dysfunction and illness. Supplying a neurotransmitter, which may be reduced in relative concentration, can often alleviate the imbalance to some extent and restore proper functioning. Other diseases, which are caused by death or dysfunction of neurons or groups of neurons, can be alleviated by supplying the neurotransmitter, which had been produced by the dead or dysfunctional neuron. Often an underlying imbalance is unknown but it has been empirically found that supplying a neurotransmitter can alleviate or ameliorate symptoms. Neurotransmitter receptor agonists or antagonists have been used successfully used to modify functioning and ameliorate symptoms. In many such cases, however, relief available from such treatment is only temporary. Due to complex self regulatory mechanisms within the brain, and to other poorly understood causes, tolerance or habitation to the receptor agonist or antagonist are often the result of treatment. Continued relief requires increasing doses of drug are required, until the treatment ceases to be effective at all or undesirable side effects come to out weigh the benefit of the treatment.

One such disease, schizophrenia, is a debilitating mental illness which affects 1% of the world's population, causes chronic disability in at least 60%-70% of affected persons, has a 10% suicide mortality risk, and still remains without known pathophysiology or etiology. Over the last four decades, the only pharmacologic treatment for schizophrenia has involved drugs which antagonize dopamine functions, mainly by blocking dopamine receptors, an approach that was serendipitously discovered in the early 1950's. The first of these therapeutic drugs for schizophrenia was chlorpromazine (Delay J, Deniker P (1952): Neurol, Le Congres Des et al., Edited by Masson, Paris.) which was identified as a dopamine receptor antagonist a decade after its clinical discovery (Carlsson A, Lindqvist L (1963): Acta Pharmacol. Toxicol. 20:140-145.). Subsequently, many dopamine receptor antagonists were developed for schizophrenia. Although almost all are effective antipsychotic agents, each has varying drug side effect profiles, predominantly parkinsonism, akathisia, and tardive dyskinesia (Klein D F, Davis J M (1969): Diagnosis and Drug Treatment of Psychiatric Disorders. The Williams and Wilkins Company, Baltimore.). No non-dopaminergic antipsychotics have been identified, although one (MDL 100 907), a pure serotonin 2a antagonist, is being tested.

At the current time, it is commonly accepted that blockade of the D₂-family dopamine receptors, inhibits dopamine-mediated neurotransmission, and thereby produces an antipsychotic response. The brain mechanisms subserving that response in humans are being studied (Holcomb H H, Cascella N G, Thaker G K, Medoff D R, Dannals R F, Tamminga C A (1996): Am J Psychiatry 153:41-49.). Anti-dopaminergic drug treatments are effective in reducing psychotic symptoms of schizophrenia (i.e. positive symptoms) but leave other aspects of the illness (e.g. negative symptoms, attention, concentration, and cognition) poorly treated. Thus, despite the partial effectiveness of current treatments, pressing need exists for new treatments.

The inventors have theorized that schizophrenic psychosis could be treated by a reduction in dopamine-mediated neurotransmission mediated by a dopamine agonist rather than through dopamine receptor blockade. In fact, dopamine agonists have been described in the literature as a means to diminish dopamine synthesis and release in experimental animals through stimulation of a negative feedback dopamine sensitive autoreceptor (Clark D, Hjorth S, Carlsson A (1985): J Neural. Transm. 62:1-52.). This idea has been successfully tested, resulting in the identification of apomorphine (a full dopamine agonist) as an antipsychotic in schizophrenia (Tamminga C A, Schaffer M H, Smith R C, Davis J M (1978): Science 200:567-568.).

The dopamine agonist with the best antipsychotic action is the partial dopamine agonist (−)-3PPP. (−)-3PPP was discovered and characterized preclinically by Dr. Arvid Carlsson and his colleagues [See U.S. Pat. No. 4,719,219]. The drug has several distinctive properties in clinical application in schizophrenia. The antipsychotic action of (−)-3PPP is broad, including both positive and negative symptoms, and is not accompanied by any acute motor side effect, no Parkinsonism, no akathisia, no dysphoria. As such, it has a very desirable therapeutic action, with far fewer side effects than any available agent. However, for full or relatively high intrinsic activity dopamine agonists, the therapeutic effect (although strong) of both of these drugs is time-limited, on the order of 1-7 days.

While not the generally accepted clinical method, treating psychosis in schizophrenia with dopamine agonists (full or partial) is not novel or unique. However, the idea has not been widely applied in clinical settings because certain dopamine agonists are known to produce tolerance, thereby attenuating the antipsychotic effect in schizophrenia. Thus, there is still a great need for a better treatment for dopamine hyperfunction disorders such as psychosis and schizophrenia.

The present invention addresses this problem: preventing the tolerance or other side effects associated with agonist therapy by means of a low dose of a dopamine antagonist. Specifically, by modifying the intrinsic activity (IA) of a dopamine agonist with concomitant dopamine antagonist dosing, the duration of drug action can be lengthened. Intrinsic activity (IA) at the dopamine receptor refers to the magnitude of agonist action of a drug delivered at its receptor, not the strength with which that drug binds to its receptor. By combining a partial dopamine agonist with dopamine receptor antagonists, in low dosage, the problem the tolerance associated with the treatment with the agonist alone (possibly, through dopamine autoreceptor down-regulation) can be overcome. Moreover, differing doses of an antagonist, such as haloperidol, can be used to adjust the IA of an agonist, such as (−)-3PPP. This latter is potentially important because, while schizophrenia treatment may require one fixed level of dopamine agonist intrinsic activity, the treatment of other hyper- or hypo-dopaminergic disorders may require different levels of agonist intrinsic activity.

A non-limiting theory of the mechanism of this effect is that the combination of (−)-3PPP with haloperidol provides such superior results because of the relatively specific action of haloperidol at the D₂ family of receptors and its relatively low other side effects at these low doses. The present invention includes the idea of combining a partial dopamine agonist, such as (−)-3PPP, and a dopamine receptor antagonist, such as haloperidol, to produce reduced intrinsic activity dopamine receptor agonism without tolerance, for the treatment of psychosis. It is clear that the present invention can also be used for the treatment of other central nervous disorders caused by dopamine dysfunction. As the newly discovered method disclosed herein acts essentially as a stabilizing procedure, it can be used in the treatment of any unstable dopamine system, for treating both hyper- and hypo- function disorders.

### SUMMARY OF THE INVENTION

It is the object of the invention to provide a pharmacologic therapy for central nervous disorders associated with receptor dysfunction wherein a partial receptor agonist is administered in combination with a full or partial receptor antagonist, the combination providing an agonist with reduced intrinsic activity. As used herein, the terms “partial agonist” and “partial receptor agonist” refer to those agents which have a strong affinity for the receptor but limited intrinsic activity. As used herein, the terms “receptor antagonist” and “full receptor antagonist” refer to agents capable of antagonizing the function of a receptor, having an intrinsic activity of zero or slightly higher.

It is a further object of the invention to provide a pharmacologic treatment for psychosis and other central nervous disorders stemming from dopamine receptor dysfunction, said treatment providing anti-psychotic effects without the physiologic side effects associated with dopamine antagonist therapy or efficacy tolerance associated with agonist therapy. The treatment involves the administration of a dopamine antagonist in combination with a partial dopamine agonist.

It is a further object of the invention to provide a drug treatment that avoids dopamine-antagonist-induced side effects, including drug-induced parkinsonism, akathisia, and tardive dyskinesia and neuroleptic-induced dysphoria, negative symptoms, and cognitive impairment.

In a preferred embodiment, the CNS disorder is schizophrenia, the dopamine agonist is (−)-3PPP, and the dopamine antagonist is haloperidol. However, the invention can be expanded to encompass combination drug therapy for other dopamine disorders including non-schizophrenic psychoses, such as dementia of the elderly, affective psychoses, such as mania and psychotic depression, episodic Axis II psychotic conditions, such as MDD and borderline conditions (Hardman, J., Gilman, A., and Limbird, L, (1995). chapters 18, 22, 24, Goodman and Gilman's The Pharmaceutical Basis of Therapeutics, McGraw Hill Co., New York.

A related object is the use of the combination drug therapy, the partial dopamine agonist together with the dopamine antagonist, to treat disorders routinely clinically treated with antipsychotic drugs, thereby reducing the motor side effects associated with dopamine antagonist drugs alone. Such disorders include but are not limited to neurodevelopmental disorders, autism, mental retardation, subclinical or latent psychosis, substance abuse alone or comorbid with schizophrenia, dementia in the elderly with behavioral disturbance, and L-DOPA-induced hallucinations in Parkinson's disease (Hardman, J., Gilman, A., and Limbird, L, (1995).chapters 18, 22, 24, Goodman and Gilman's The Pharmaceutical Basis of Therapeutics, McGraw Hill Co.,New York.

### BRIEF DESCRIPTION OF THE DRAWINGS

FIG. 1: Depicts the in vitro effect of the dopamine antagonist haloperidol on the intrinsic activity of partial dopamine agonist, (−)-3-PPP.

### DETAILED DESCRIPTION OF THE INVENTION

Standard drug therapy for psychosis and other dopamine receptor dysfunctional involves continuous treatment with regular doses of dopamine receptor antagonists. While this therapy is often at least partially effective for the treatment of the positive symptoms, namely psychosis, it fails to address other aspects of the illness, namely the negative symptoms, cognition, attention and concentration. Additionally, all anti-dopaminergic drugs, currently available, have certain significant side effects, including parkinsonism, akathisia, and tardive dyskinesia. At present, there are research treatments that involve administration of dopamine agonists. As mentioned above, this treatment suffers from efficacy tolerance. Therefore, it is clear there is a need for effective antipsychotics that treat both positive, cognitive, and negative symptoms of the disorder without the commonly reported motor side effects.

The object of the invention is to allow long term treatment of a disease of the central nervous system by receptor agonists, without development of tolerance to the treatment. By long term treatment or therapy, it is meant treatment with no predetermined end point, such as might continue for weeks or months, or even for the life of the patient. The basis of the current invention was the insight that lowering the agonist intrinsic activity (IA) might be a mechanism to decrease efficacy tolerance to the drug. It has been recently discovered that mixtures of a full antagonist with a partial agonist, each for the same neurotransmitter system, can result in a reduced IA, partial agonist. In preliminary studies, the inventors discovered that partial agonists with lower IA have a longer duration of clinical antipsychotic action. Based on this effect, the goal was to expand the duration of effect of a desirable dopamine agonist treatment for schizophrenia and convert it into a therapeutically viable treatment by reducing the agonist IA through combining it with a very low dose of a full dopamine antagonist. In initial experiments, the agonist used was (−)-3PPP and the antagonist used was haloperidol. The results of these experiments, discussed in detail below, confirmed the basic premise, that a low dose of an antagonist delivered in combination with a dose of partial agonist can preserve the anti-psychotic activity and lengthen our indefinitely the therapeutically beneficial properties of the agonist.

The present invention is the first to knowingly combine an agonist with varying concentrations of its own receptor's antagonist. This combination has the effect of modifying the intrinsic activity of the agonist for treatment of a human disease. Although the preferred combination of drugs targets the dopamine receptor, these principles and strategies would be similarly applicable to other CNS transmitter systems and disease treatments.

The present invention represents a pharmacologic therapy that is vastly superior to any that are currently applied in the clinical setting or even contemplated by the literature. The advantage of such combination drug therapy in the field of schizophrenia is the equivalent treatment of positive psychotic symptoms, without any motor or dysphoric side effects, no elevation of plasma prolactin levels, and with potential for the treatment of negative and cognitive symptoms of this illness. Negative symptoms, when untreated in schizophrenia, markedly impair psychosocial recovery and rehabilitation. No current antipsychotics effectively treat negative symptoms, making the identification of an effective agent imperative. The motor side effects of current antipsychotic treatments are extremely painful and bothersome to the patient. These side effects also reduce compliance and are consequently associated with drug discontinuation and illness relapse. So antipsychotic treatments without motor side effects are important and clinically relevant. Moreover, the disclosed drug combination can reduce the long term incidence of the delayed neuroleptic-induced motor side effect called, tardive dyskinesia. The medical and legal implications of this possibility are considerable.

Clearly, the potential advantages of this treatment over other currently available treatments are great. Because the treatment for schizophrenia is a receptor agonist, this treatment strategy avoids dopamine-antagonist-induced side effects: these include drug induced parkinsonism, akathisia, and tardive dyskinesia; they can also include neuroleptic-induced dysphoria, negative symptoms, and cognitive impairment. Moreover, the drug combination of the present invention can serve as a primary treatment for negative symptoms in schizophrenia and for cognitive dysfunction in the illness. In other conditions, especially affective psychosis (mania and psychotic depression), the drug therapy of the instant invention can provide effective treatment without any tardive dyskinesia risk. The limited success of antipsychotic treatments in the non-schizophrenic psychoses is in large part due to neuroleptic-induced side effects, which could be avoided with this combination.

As the molecular structures of the different dopamine (DA) receptors has been defined over the last decade (Gingrich, J. A., Caron, M. G.: Annual Review of Neuroscience, (1993) 16:299-321. Jarvie, K. R., Caron, M. G.: Advances in Neurology, (1993) 60:325-333.), the action of drugs at the different receptor sites has become increasingly clear. Multiple DA receptors exist, as multiple targets for drug action. The structure of all DA receptors is a G protein-coupled receptor, where the seven transmembrane-spanning sites are arranged in a donut-like shape in the membrane. How a drug interacts with a receptor, to deliver its action, is becoming increasingly clear. The sites where DA electrostatically binds to the critical amino acids to effect a change in protein conformation are known: Asp₃, Ser₅ and Ser₅. Agonists affect the protein conformational change at the D₂ receptor with different potencies and consequently deliver different potency signals to the cell. Antagonists merely cap the top of this receptor complex, without producing any effect on the receptor structure at all (Tamminga, C. A., Dahl, S. G. (1994) Am. J. Psychiatry, 151:4.).

The use of a receptor agonist is different in several practical respects from a receptor antagonist. Since a receptor antagonist blocks all (or nearly all) transmission at that receptor, any drug-induced alterations in that receptor are not an immediate issue for the nature of clinical drug action. However, an agonist changes the state of the receptor by reason of its receptor activation, producing receptor desensitization. If continued therapeutic stimulation of the receptor is desired, special techniques are necessary to avoid desensitization and preserve activity. Repeated agonist stimulation of a receptor produces desensitization and ultimate insensitivity of that receptor to the stimulus. This is physiologic, and occurs in all brain, G protein systems. The details of the molecular events of receptor desensitization are currently being studied (Gingrich, J. A., Caron, M. G. (1993) Annual Review of Neuroscience, 16:299-321; Jarvie, K. R., Caron, M. G. (1993) Advances in Neurology, 60:325-333; Caron, M. G. (1994): in Dopamine Receptor Subtypes in Neurological and Psychiatric Diseases, Brooklodge, Kalamazoo, Mich.). Agonist stimulation at any G protein- coupled recognition site produces phosphorylation of amino acids near the distal end of the third intracytoplasmic loop. This phosphorylation putatively facilitates the attraction between the recognition site and the effector protein of the receptor and is responsible for receptor desensitization, then receptor involution, and finally actual intracellular breakdown of the receptor protein (Caron, M. G. (1994): in Dopamine Receptor Subtypes in Neurological and Psychiatric Diseases, Brooklodge, Kalamazoo, Mich.). There are known medical diseases linked to abnormalities of receptor desensitization, suggesting the physiologic and pathophysiologic importance of this process (Lefkowitz, R. J. (1993) Nature, 365:603-604,.). Again, while not wishing to be bound by theory, tolerance to the clinical therapeutic actions of (−)-3PPP is most likely based on this desensitization response to agonist stimulation.

Partial agonists at any receptor are those drugs which have a strong affinity for the receptor but limited intrinsic activity. Partial agonists are attracted to a receptor and bind to it, with an affinity similar to the natural ligand, but, once bound, have a lesser activity (Ariens, E. J. (1954) Arch. Int. Pharmacodyn. Ther., 99:32-49, Kenakin, T. P. (1993) Pharmacologic Analysis of Drug-receptor Interaction. Raven Press, New York.). Depending on the state of occupancy of that receptor, and the drug's intrinsic activity, these partial agonists can have an overall antagonist or agonist action on neurotransmission at that synapse. Partial agonists can have a high level of intrinsic activity (approaching the 100% activity of the natural agonist) and act much like a full agonist; or, they can have a low level of intrinsic activity (perhaps of 5% -10%) and act nearly like an antagonist. In between, partial agonists have widely varying levels of intrinsic activity, and differing behavioral actions. The resultant pharmacologic action of these medium-intrinsic activity agonists depends on the state of the target system. Over the last twenty years, considerable work has been done testing dopamine agonists in schizophrenia (Tamminga C A, Schaffer M H, Smith R C, Davis J M (1978): Science 200:567-568; Tamminga C A, Gotts M D, Thaker G K, Alphs L D, Foster N L (1986): Arch Gen Psychiatry 43:398-402; Corsini, G. U., DelZompo, M., Manconi, S., Cianchetti, C., Mangoni, A., Gessa, G. L.: (1977) Adv Biochem Psychopharmacol, 16:645-648; Corsini, G.U., Pitzalis, G. F., Bernardi, F., Bocchetta, A., Del Zompo, M. (1981) Neuropharnacol, 20:1309-1313; Ferrier, E. C., Johnstone, E. C., Crow, T. J. (1984) Br J Psychiatry, 144:341-348; but only recently have partial agonists been available for clinical study (Olbrich, R., Schanz, H.: (1988) Pharmacopsychiat., 21:389-390, Winckler, P., Bartels, M.: Psychiatric University Hospital Tuebingen (FRG); Benkert, O., Grunder, G. Wetzel, H.: (1992) Pharmacopsychiatry, 25:6; Murasaki, M., Miura, S., Ishigooka, J., Ishii, Y., Takahashi, A., Fukuyama, Y.: (1988) Prog. Neuro-Psychopharmacol. & Biol. Psychiat., 12:793-802; Kiuchi, K., Hirata, Y., Minami, M., Nagatsu, T.: (1988) Life Sciences, 42:343-349.). Of the partial agonists studied recently, (−)-3PPP is the only one which is relatively selective for dopamine receptors and lacks activity at other monoamine receptors (Hjorth, S., Carlsson, A., Clark, D., Svensson, K., Wikstrom, H., Sanchez, D., Lindberg, P., Hacksell, U., Arvidsson, L. E., Johansson, A., Nilsson, J. L. G. (1983) Psychopharmacol., 81:89-99).

Until recently, attempts to identify additional agonists as antipsychotic have not yielded good results. It is possible that the failures result from the broad monoaminergic action of these non-aporphine dopamine agonists and their high level of intrinsic activity. As discussed above, intrinsic activity (IA) at the dopamine receptor refers to the magnitude of agonist action of a drug delivered at its receptor, not the strength with which that drug binds to its receptor. Subsequently, we have identified two other dopamine agonists, n-propylnorapomorphine (NPA) (Tamminga C A, Gotts M D, Thaker G K, Alphs L D, Foster N L (1986): Arch Gen Psychiatry 43:398-402.) and (−)3-(3-hydroxyphenyl)-N-n- propylpiperidine [(−)-3PPP)] (Lahti A C, Weiler M A, Corey P K, Lahti R A, Carlsson A, Tamminga C A (1997): In Press, Biological Psychiatry.) as drugs demonstrating antipsychotic properties.

By better understanding the relationship between intrinsic activity and receptor manipulation, it will be possible to more efficiently design and discover novel, unique therapeutic agents that can treat disorders associated with transmitter malfunction. In the past, an agent determined to have too high of an intrinsic activity for a particular therapy was discarded, forcing the scientist to go back to the “drawing board” to design another drug. Now, using the information and techniques disclosed herein, it will be possible to reevaluate those agents determined to be ineffective or inefficient from a therapeutic perspective. Many transmitter affecting agents previously rejected, having been tested only in as a single agent drug therapy, may find utility when used in combination with counter-agents of same neurotransmitter system.

While the invention has been described in detail, and with reference to specific embodiments thereof, it will be apparent to one with ordinary skill in the art that various changes and modifications can be made therein without departing from the spirit and scope thereof. It is clear that this drug combination could also be applied to other mental or motor conditions, including affective and anxiety disorders (mania or depression), psychosis of the elderly with or without dementia, and in episodic Axis II psychotic conditions like multiple personality disorders and borderline conditions (Hardman, J., Gilman, A., and Limbird, L, (1995). chapters 18, 22, 24, Goodman and Gilman's The Pharmaceutical Basis of Therapeutics, McGraw Hill Co., New York). Because of the very low risk (if any) of tardive dyskinesia with this treatment, it would reduce the medical and legal risk associated with current antipsychotic treatments especially as those are manifest in non-schizophrenic psychosis where the incidence of tardive dyskinesia is greater. Moreover, this treatment could also be applied in neurologic diseases or disorders, including motor disorders, where dopamine dysfunction is involved and neuroleptics or dopamine agonists are now used. Such diseases or disorder include but are not limited to, for example in Tourette's disease, Huntington's chorea, Parkinson's Disease, tardive dyskinesia, obsessive-compulsive disorder. A current treatment for Parkinson's Disease is carbidopa-levodopa, such as Sinemet®, which is dosed at 300-10000 mg bid. Could be combined with a dopamine antagonist such as holoperidol to prevent tolerance or side effects.

Likewise, this treatment could be used to treat disorders currently treated with antipsychotic drugs for clinical reasons. Such disorders include but are not limited to neurodevelopmental disorders and autism, mental retardation, subclinical or latent psychosis, schizophrenia comorbid with substance abuse, substance abuse alone, dementia in the elderly with behavioral disturbance, and L-DOPA-induced hallucinations in Parkinson's disease. Patients with such diseases or disorders currently being treated with antipsychotic drugs would especially benefit from the “no-motor-side-effect” profile of this drug combination. Furthermore, as the biochemical principles are essentially the same for most G protein receptors, one could utilize the teachings herein to provide a pharmacologic therapy for central nervous disorders associated with the dysfunction of other transmitters, such as serotonin, norepinephrine, or acetyl choline. A specific pharmacologic treatment or therapy would involve the administration of a full or partial receptor agonist in combination with a receptor antagonist, the combination providing an agonist with reduced intrinsic activity. For drugs for which specific doses are not given in the examples, the general dosing schedule would be to dose the receptor antagonist at approximately one tenth normal dose, and the agonist at normal dose levels, starting at a low dose and increasing as would be normal in clinical practice.

It is within the scope of the invention to formulate or compound any single receptor agonist or receptor antagonist or combination thereof so as to provide a pharmaceutically acceptable dosage form. Such a dosage form could comprise an oral or parenteral dosage. The drug could be administered as a single dose or multiple doses, containing single or more than one active drug. The dose range for each component is within the competence of one skilled in the art to determine for each patent. The skilled practitioner would consider the recommended dose of the drug, the condition of the patent, and the tractability of the symptoms to therapy. The skilled practitioner would adjust the dose over a period of days to weeks to achieve maximum efficacy. Practitioners in psychiatric and neurologic practice routinely perform such dose adjustment as part of normal practice.

All publications cited herein are incorporated by reference in their entirety. cl EXAMPLES

The data herein can predict and help select optimal agonist intrinsic activity for clinical testing for anti-psychotic activity, duration of therapeutic action, and side effects. Relative dosages of partial agonist and full antagonist can be determined using routine experimentation. However, the following examples are provided for illustrative purposes only, and are in no way intended to limit the scope of the present invention.

### Example 1

### Dopamine Stimulation and Inhibition of Tardive Dyskinesia

It is clear that there is a relationship between agonist therapy and tardive dyskinesia inhibition. Studies of partial dopamine agonist, (−)-3PPP, were extended to additional syndromes sensitive to anti-dopaminergic strategies. Tardive dyskinesia (TD) is the hyperkinetic involuntary movement disorder, which occurs in the context of chronic neuroleptic treatment. It occurs at an incidence rate of 5% per treatment year, determined prospectively (Kane J M, In: Bloom F E and Kupfer D J (Eds) Psychopharmacology: The Fourth Generation of Progress. Raven Press, New York, pp. 1485-1495, 1995.). Since overall prevalence rates are 20%-60%, the expectation is that the incidence rate will plateau after some number of treatment years, but the inflection point is not yet clear. While the etiology of TD is clear (i.e. chronic neuroleptic administration), the exact pathophysiology, necessary to target prophylaxis or treatment, is unknown.

The clinical pharmacology of TD suggests the utility of drugs, which decrease DA-mediated neurotransmission (e.g. partial DA agonists) or GABA agonists as therapeutically effective. Some data from human (Thaker G K, Tamminga C A, Alphs L D, Lafferman J, Ferraro T N, Hare T A: (1987) Arch. Gen. Psychiatry 44:522-529.) and from animal studies (Kaneda H, Shirakawa O, Dale J, Goodman L, Bachus S E, Tamminga C A: (1992) Eur J Pharmacol 212:43-49) both suggest that dyskinesia can be reversed in TD.

The clinical pharmacology of dopamine system stimulation and inhibition in tardive dyskinesia is surprisingly similar to schizophrenia. Dopaminergic stimulation with potent indirect agonists characteristically worsen tardive dyskinesia. D-amphetamine clearly worsens dyskinesia in a dose-dependent fashion; bromocriptine probably worsens dyskinesia even though not significantly (Tamminga C A, Chase T N: (1980) Arch Neurol 37:204-205). As in psychosis, apomorphine paradoxically improves tardive dyskinesia, both significantly and substantially (Tamminga C A, Schaffer M H, Smith R C, Davis J M (1978): Science 200:567-568.). CF25397 (a lower affinity ergot dopamine agonist) and piribedil (a full DA agonist) modestly improved dyskinetic symptoms in TD (Table 1) (Tamminga, C. A.: Ergot Compounds and Brain Function. In: M. Goldstein et al. (Eds.) Neuroendocrine and Neuropsychiatric Aspects. Raven Press, New York, 1980.). Moreover, (−)-3PPP improves L-DOPA-induced dyskinesia in Parkinson's disease, a motor condition in some ways similar to TD.

TABLE 1
Comparative Properties of Dopamine Agonists (87)

| | Contraversive | Stereotyped | Adenylate | Tyrosine | Antidys- | Anti- |
|---|---|---|---|---|---|---|
| Drug | Turning | Behaviors | Cyclase | Hydroxylase | kinetic | psychotic |
| Apomorphine | +++ | +++ | +++ | +++ | ++ | ++ |
| Bromocriptine | ++ | ++ | −−− | ND² | −− | − |
| CF25-397 | + | −− | + | ND | ± | 0 |
| Lisuride | +++ | ++ | −−− | −−− | ND | + |
| Lergotrile | +++ | ++ | −−− | −−− | ND | −− |
| Piribedil (S584) | ++ | ++ | ++ | +++ | + | 0 |

^{a}ND, no data

### Example 2

### Testing -3PPP Tolerance Levels

In the course of studying dopamine agonists for the treatment of psychosis, assayed dopamine agonists to determine tolerance limits. Intrinsic activity is determined using a functional assay which involves an agonist-induced release of [³H]arachidonic acid from CHO cells stably transfected with the D₂ receptor (Lahti, R. A., Figur, L. M., Piercey, M. F., Ruppel, P. L., Evans, D. L.: (1992) Mol. Pharmacol., 42:432-438). Cloned dopamine receptors are used to quantify intrinsic activity , with the intrinsic activity estimates calculated using the ratio of the compounds affinity at the low-affinity agonist state to its affinity for the high affinity agonist state.

First, in examining the efficacy tolerance of NPA, a full dopamine agonist (IA=90%), tolerance was observed after 24 hours. Next, the efficacy tolerance of (−)-3PPP, the partial dopamine agonist (IA=40%), produced a longer efficacy, lasting up to 7 days.

The observation that lower intrinsic activity seemed to relate to longer therapeutic effect gave an indication as to how to extend the therapeutic efficacy of partial agonists like (−)-3PPP and block tolerance. This initial observation in conjunction with the known fact that mixtures of full antagonists with partial agonists, each for the same neurotransmitter system, results in reduced IA partial agonist led the inventors to perform the subsequent experiments described herein to confirm the theory.

### Example 3

### Screening Drugs for “Ideal” Partial Agonist Characteristics

Many potential compounds were screened in this model to find drugs with favorable partial agonist characteristics for treating psychosis. These characteristics include: an activity restricted to the dopamine D₂ system, with little affinity for the D_{1A} or serotonin 5HT_{1A} or 5HT_{2A} receptors, and an intrinsic activity between 18% and 35%. Examples of data generated in the evaluation of a number of compounds are presented in the following three tables (Tables 2-4).

TABLE 2
Affinities and Intrinsic Activities of Dopamine Agonists

| | hO₃ Receptor | | | hO₄₄ Receptor | | |
|---|---|---|---|---|---|---|
| | | Ratio | Intrinsic | | Ratio | Intrinsic |
| Compound | Ki (nM) ± S.D | Low/High | Activity | Ki (nM) ± S.D. | Low/High | Activity |
| Dopamine | 4.5 ± 0.6 | 463 | 100% | 3.9 ± 0.7 | 471 | 100% |
| (−)-Apo | 1.5 ± 1.1 | 124 | 91% | 0.3 ± 0.2 | 64 | 80% |
| (+)-Apo | 15.0 ± 9.1 | 47 | 75% | 3.0 ± 1.5 | 15 | 55% |
| (−)-3-PPP | 30.9 ± 13.4 | 9 | 46% | 12.2 ± 3.1 | 77 | 83% |
| (+)-3-PPP | 66.4 ± 12.5 | 68 | 81% | 12.0 ± 6.5 | 164 | 96% |
| (−)-NPA | 0.07 ± 0.04 | 208 | 100% | 0.6 ± 0.2 | 17 | 57% |
| (+)-NPA | 14.0 ± 5.0 | 21 | 61% | 2.3 ± 2.0 | 18 | 58% |
| (−)-N-0437 | 0.08 ± 0.04 | 145 | 94% | 1.1 ± 0.7 | 106 | 88% |
| (+)-N-0437 | 4.8 ± 2.3 | 10 | 48% | 7.7 ± 3.4 | 40 | 72% |

TABLE 3
Affinity and Intrinsic Activity of the Partial Dopamine Agonists
FCE23884, CL-1007 and DUP-127090 for the hD₂₁ Receptor

| | | | Intrinsic |
|---|---|---|---|
| Compound | Kᵢ (HiAg State) | Kᵢ (LowAg State) | Activity |
| CL-1007 | 0.48 nM | 5.4 nM | 49.5% |
| DUP-127090 | 3.04 nM | 6.41 nM | 21.0% |
| FCE-23884 | 0.40 nM | 2.17 nM | 37.0% |

TABLE 4
Summary of Affinities and Intrinsic Activities of Dopamine
and (−)-3-PPP for hD₂₁ and hD₄-type Receptors

| | |
|---|---|
| Dopamine | (−)3-PPP |

| | | Intrinsic | | Intrinsic |
|---|---|---|---|---|
| Receptor Type | Kᵢ (nM)^{#} | Activity % | Kᵢ (nM)^{#} | Activity % |
| hD₃ | 4.5 | 100% | 30.9 | 46% |
| hD₄₂ | 6.0 | 100% | 30.9 | 73% |
| hD₄₄ | 2.7 | 100% | 13.2 | 97% |
| hD₄₇ | 2.4 | 100% | 14.3 | 98% |

^{#}Affinity was determined at the high affinity agonist state using a [³H]-aconist ligand.

### Example 4

### Receptor Desensitization

It is possible to experimentally predict the level of IA of an agonist which will be devoid of any receptor down-regulating properties. The basic premise of the present invention is that reduction of the agonist IA will tend to reduce tolerance, and ultimately obliterate it entirely. Tolerance likely occurs because of receptor desensitization in vivo. Thus, laboratory studies of drug concentrations associated with no desensitization help to rationally to select and test partial dopamine agonists and/or their combinations.

The preferred technique for assaying the dopamine receptor sensitivity and regulatory effects was used. Specifically, it has been shown that chronic treatment of CHO cells expressing the cloned 5HT_{1A} receptor with a 5HT_{1A} agonist results in the down-regulation and functional desensitization of the 5HT_{1A} receptor (Rotondo A, Nielsen D A, Nakhai B, Hulihan-Giblin B, Bolos A, Goldman D. (1997). Neuropsychopharmacolgy 17: 18-26.). Since the D₂ receptor is similar to the 5HT_{1A} receptor in that it is coupled to G₁, chronic D₂ agonist treatment of CHO cells was used in a parallel technique to study receptor down-regulation. The full agonist U-86170, which is stable in aqueous solutions was used, in various doses to desensitize the CHO cell containing D₂ receptor, in comparison to dopamine. Once the “ideal” intrinsic activity is arrived at, it can be used to study the agonist (−)-3PPP alone and in combination with varying doses of haloperidol or clozapine to determine the relationship between IA and D₂ receptor desensitization.

### Example 5

### Testing the Agonist/Antagonist Activity

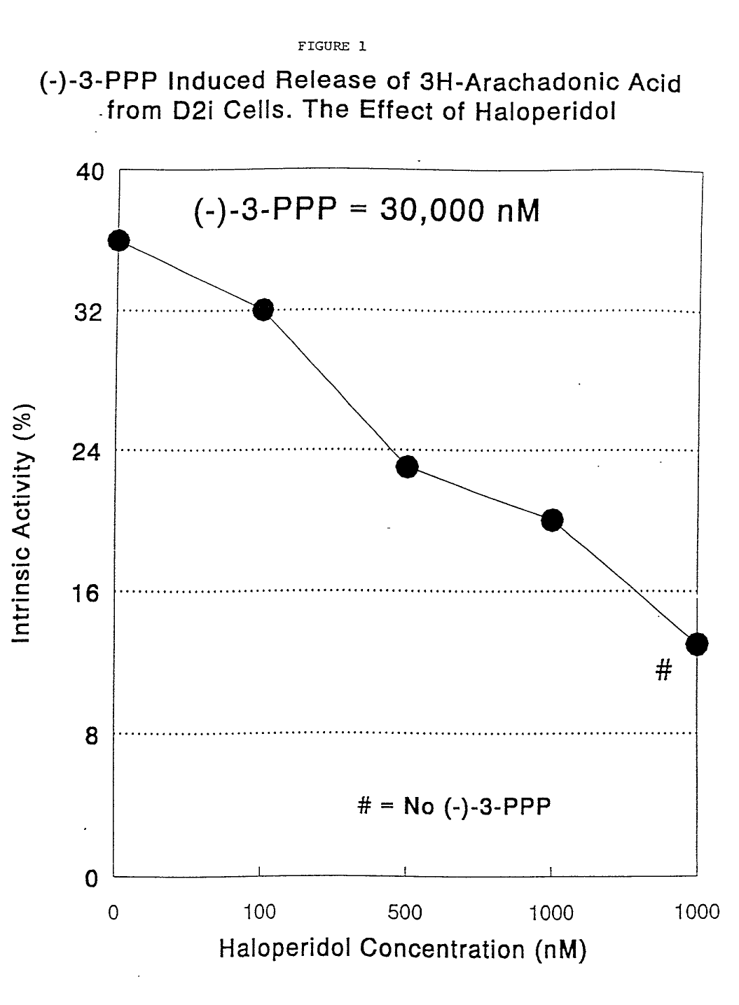

Combining a receptor agonist with its own antagonist can reduce the agonist's intrinsic activity. In the laboratory, this was shown for (−)-3-PPP. It was also found haloperidol can reduce the intrinsic activity of (−)3-PPP from 36%, when it is tested without haloperidol, to approximately 20% intrinsic activity in the presence of 1000 nM haloperidol, FIG. 1. Thus a 30:1 ratio of (−)-3-PPP to haloperidol would produce a designed mix with an intrinsic activity of almost one-half that of the partial agonist alone. These data confirm the idea that combining (−)-3-PPP with low concentrations of a full antagonist, like haloperidol, will reduce its functional intrinsic activity. (−)-3PPP was combined with varying concentrations of haloperidol and demonstrated a linear reduction in the intrinsic activity of (−)-3-PPP with increasing concentrations of haloperidol (FIG. 1).

After demonstrating the antipsychotic action of (−)-3-PPP, but showing that it has only a duration of approximately one week with continuous dosing (Lahti A C, Weiler M A, Corey P K, Lahti R A, Carlsson A, Tamminga C A (1997): In Press, Biological Psychiatry.), a combination of (−)-3-PPP and a low dose of a known dopamine receptor antagonist, haloperidol was tested. This combination was designed to produce a lower IA (−)-3PPP, and thereby prolonging its antipsychotic activity. To test this, actively psychotic schizophrenic persons were tested with (−)-3PPP (300 mg bid) plus haloperidol (1 mg bid), compared to the haloperidol alone. This dose of haloperidol may have delivered a measurable antipsychotic effect. However, the antipsychotic effect of (−)-3PPP was apparent and was present in week one and it extended longer for the entire two week period of the trial without evidence of tolerance. (Table 5). These data support the basic premise that combination of a low dose of antagonist with (−)-3PPP will preserve its antipsychotic activity and lengthen out perhaps indefinitely the action (−)-3PPP.

TABLE 5
Change In BPRS Score With (−)-3PPP (300 mg bid) Plus
Haloperidol (1 mg bid) Over Two Weeks of Treatment compared
With Placebo (bid) Plus Haloperidol (1 mg bid) ForTwo Weeks.
Total BPRS Score

| | |
|---|---|
| (−)-3PPP | Placebo |

| Rating Time | mean | SD | Mean | SD |
|---|---|---|---|---|
| Baseline | 38.5 | 11.1 | 38.5 | 11.1 |
| Day 3 | −1.4 | 3.6 | −1.0 | 5.8 |
| Day 7 | −3.4 | 3.2 | −0.2 | 5.5 |
| Day 11 | −3.3 | 4.2 | −0.1 | 6.1 |
| Day 14 | −4.6 | 4.3 | −1.1 | 5.0 |

### Example 6

### Clinical Trial of Combination Therapy Effacacy

A preliminary clinical trial co-administration of receptor agonist and antagonist is being conducted. The efficacy of treatment is further tested by comparing placebo treated to drug treated groups. The study is designed to demonstrate that co administration of the dopamine antagonist, haloperidol with the partial receptor agonist, (−)-3-PPP reduces symptoms of psychosis.

Study Design: Patients are entered into the study based of psychiatric evaluation based on at least weekly ratings. After being entered into the study patients are randomly assigned into experimental or control groups. The study is conducted double blind so that neither patients nor physician are aware of which group patients are entered into. Patients are treated with haloperidol 0.5 mg b.i.d, for two weeks to establish stable conditions. Patients are then treated with the partial dopamine receptor agonist, (−)-3-PPP, at a dose between 300 and 600 mg bid. The dose of (−)-3-PPP is corrected over the first week depending on the evaluation of the psychiatrist. Optimal dose levels are often approximately 450 mg bid. Patients receive haloperidol at a constant dose throughout. Control groups receive haloperidol and a placebo tablet in place (−)-3-PPP. Groups are maintained in treatment for 4-6 weeks, with continued weekly rating.

A minimum of ten patients will be randomized to each group. Patients will be evaluated weekly by a rating system to measure improvement.

### Definitions

Receptor:

a protein with a distinctive tertiary structure, often membrane bound, with specialized areas for recognizing particular signaling molecules on a ligand; when a receptor complexes with its ligand the interaction alters subsequent cellular properties (electrophysical, biochemical, and/or molecular).

Neurotransmitter:

a signaling substance within nervous system which transmits encoded signals to neurons at a specialized receptor site.

Agonist:

a signaling chemical which, when coupled to its specific receptor, effects a characteristic cellular action.

Antagonist:

a chemical substance which couples to a specific receptor without producing an action except to block other substances from that receptor.

D₂ dopamine receptor:

a cell-surface G protein coupled receptor protein which recognizes dopamnine as its natural neurotransmitter and shows a high affinity for such ligands as reclopride and haloperidol but not SCH 23390; highly concentrated in basel ganglia.

Psychosis:

a state of reality distortion where the psychotic person has perceptual experiences not supported by reality, imagines malice falsely and/or has involuntary, disconnected, illogical thoughts.

What is claimed:

1. A method for treating a central nervous disorder associated with dopamine dysfunction comprising:
(a) administration of a full or partial dopamine receptor agonist, to be administered in a amount reduce symptoms of said disorder, and
(b) co-administration of a dopamine receptor antagonist, said dopamine receptor antagonist being administered in an amount sufficient to reduce the intrinsic activity of the receptor agonist.

2. The method of claim 1, wherein said receptor antagonist is haloperidol.

3. The method of claim 2, wherein haloperidol is administered in a dose between about 0.1 mg and 1.5 mg, b.i.d.

4. The method of claim 2, wherein the haloperidol is administered in a dose between about 0.2 mg and 1 mg b.i.d.

5. The method of claim 2, wherein the haolperidol is administered in a dose between about 0.3 and 0.7 mg b.i.d.

6. The method of claim 1, wherein said receptor antagonist is clozapine.

7. The method of claim 6, wherein the clozapine is administered in a dose between about 10 mg and 80 mg b.i.d.

8. The method of claim 6, wherein the clozapine is administered in a dose of between about 20 mg and 60 mg. b.i.d.

9. The method of claim 1, wherein said receptor agonist is (−)-3-PPP.

10. The method of claim 9, wherein the (−)-3-PPP is administered in a dose between about 200 and 800 mg b.i.d.

11. The method of claim 9, wherein the (−)-3-PPP is administered in a dose between about 300 and 600 mg b.i.d.

12. The method of claim 9, wherein the (−)-3-PPP is administered in a dose between about 400 and 500 mg b.i.d.

13. The method of claim 1, wherein said receptor agonist is apomorphine.

14. The method of claim 13, wherein the apomorphine is administered in a dose between about 0.2 mg and 6 mg b.i.d.

15. The method of claim 13, wherein the apomorphine is administered in a dose of between about 0.4 mg and 4 mg b.i.d.

16. The method of claim 13, wherein the apomorphine is administered in a dose between about 0.5 and 3 mg b.i.d.

17. The method of claim 1, wherein said receptor agonist is NPA.

18. The method of claim 1, wherein said receptor antagonist is one of the group of bromocryptine, lisuride, CF25397, lergotrile, and piribedil.

19. The method of claim 1, wherein said central nervous disorder is psychosis.

20. The method of claim 19, wherein said psychosis is schizophrenia.

21. The method of claim 1, wherein said central nervous disorder is a motor disorder.

22. A method for treating a central nervous system transmitter receptor disorder, said receptor comprising a receptor utilizing G proteins, comprising:
(a) administering a partial or full receptor agonist and
(b) co-administering a receptor antagonist, said receptor antagonist modifying said receptor agonist intrinsic activity.

23. The method of claim 22, wherein said agonist receptor agonist is (−)-3-PPP

24. The method of claim 22, wherein said (−)-3-PPP is administered in a dose of between about 300 and 600 mg b.i.d.

25. The method of claim 22, wherein said (−)-3-PPP is administered in a dose between about 400-500 mg b.i.d.

26. The method of claim 22, wherein said receptor agonist is apomorphine.

27. The method of claim 22, wherein the receptor agonist is NPA.

28. The method of claim 22, wherein said receptor antagonist is one of the group of haloperidol and clozapine.

29. The method of claim 22, wherein said receptor is a dopamine receptor.

30. The method of claim 22, wherein said receptor is a serotonin receptor.

31. The method of claim 22, wherein said receptor is a norepinepinine receptor.

32. The method of claim 22, wherein said receptor is an acetyl choline receptor.

33. A pharmaceutical composition for the treatment of a disorder of the central nervous system; said disorder being treated by a modification of a dopamine receptor; said composition being administered at substantially the same dose during long term therapy comprising: a full or partial dopamine receptor agonist co administered with a dopamine receptor antagonist.

34. The pharmaceutical composition of 33, wherein the partial dopamine receptor agonist is (−)-3-PPP.

35. The pharmaceutical composition of 33, wherein the partial dopamine receptor is apomorphine.

36. The pharmaceutical composition of 33, wherein the partial dopamine receptor is NPA.

37. The pharmaceutical composition of claim 33, wherein the dopamine receptor antagonist is haloperidol.

38. The pharmaceutical composition of claim 33, wherein the dopamine receptor antagonist is chlorpromazine.

39. The pharmaceutical composition of claim 33, wherein the central nervous system disorder is psychosis.

40. The pharmaceutical composition of claim 33, wherein the central nervous system disease is a movement disorder.
